(19) United States
(12) Patent Application Publication
(10) Pub. No.: US 2026/0077186 A1
(43) Pub. Date: Mar. 19, 2026

(54) CARDIAC LEAD WITH BRAIDED COIL LINER

(71) Applicant: Cardiac Pacemakers, Inc., St. Paul, MN (US)

(72) Inventors: Andrew L. De Kock, Ham Lake, MN (US); Jonathan Newinski, Minneapolis, MN (US); Alex Felber, Prior Lake, MN (US)

(21) Appl. No.: 19/327,580

(22) Filed: Sep. 12, 2025

Related U.S. Application Data
(60) Provisional application No. 63/694,694, filed on Sep. 13, 2024.

(51) Int. Cl. A61N 1/05

(52) U.S. Cl. CPC A61N 1/0573

(57) ABSTRACT

An implantable lead for use with an implantable medical device (IMD) includes a tubular lead body, a proximal connector at the proximal end of the lead body configured for mechanically and electrically coupling the lead to the IMD, a helical electrode extending distally from a distal tip of a distal region of the lead body, a braided liner positioned in a first lead body lumen in the tubular lead body, and a first electrical conductor extending through a liner lumen in the braided liner.

### CROSS REFERENCE TO RELATED APPLICATIONS

This application claims priority to U.S. Provisional Patent Application No. 63/694,694 entitled “CARDIAC LEAD WITH BRAIDED COIL LINER,” filed Sep. 13, 2024, which is hereby incorporated by reference in its entirety.

### TECHNICAL FIELD

The present disclosure relates to medical electrical leads and associated manufacturing methods and methods of use. In particular, the present disclosure relates to implantable medical electrical leads for stimulating the conduction system of the heart.

### BACKGROUND

Various types of medical electrical leads for use in cardiac rhythm management (CRM) and neurostimulation systems are known. For CRM systems, such leads are typically extended intravascularly to an implantation location within or on a patient's heart and thereafter coupled to a pulse generator or other implantable device for sensing cardiac electrical activity, delivering therapeutic stimuli, and the like. In some instances, the leads need to be removed from a patient after many years of service, and maintaining structural integrity of the lead is desirable so that the lead can be completely extracted in one piece.

### SUMMARY

In Example 1, an implantable lead for use with an implantable medical device (IMD), the implantable lead comprising: a tubular lead body having a proximal end and a distal end, a proximal connector at the proximal end of the lead body configured for mechanically and electrically coupling the lead to the IMD, a helical electrode extending distally from a distal tip of a distal region of the lead body, a braided liner positioned in a first lead body lumen in the tubular lead body, and a first electrical conductor extending through a liner lumen in the braided liner.
In Example 2, the implantable lead of Example 1, wherein the braided liner comprises a plurality of ribbons that are braided together to form a tube.
In Example 3, the implantable lead of Example 2, wherein the braided liner is composed of between eight and sixty-four ribbons.
In Example 4, the implantable lead of any of Examples 2 or 3, wherein the braided liner is composed of eight ribbons woven in a one-over-one-under pattern.
In Example 5, the implantable lead of any of Examples 2-4, wherein the braided liner is woven in a one-over-one-under pattern.
In Example 6, the implantable lead of any of Examples 2-5, wherein the braided liner is woven at forty-six to fifty-two pics per inch.
In Example 7, the implantable lead of any of Examples 2-6, wherein each of the plurality of ribbons comprises primarily a polyimide material or a polyethylene terephthalate (PET) material.
In Example 8, the implantable lead of Example 7, wherein each of the plurality of ribbons does not contain fluoropolymer materials.
In Example 9, the implantable lead of any of Examples 1-8, wherein the braided liner completely covers a portion of an exterior of the first electrical conductor.
In Example 10, the implantable lead of any of Examples 1-8, wherein the braided liner only partly covers a portion of an exterior of the first electrical conductor.
In Example 11, the implantable lead of any of Examples 1-10, wherein: the tubular lead body further comprises a second lead body lumen extending from the proximal end through the distal end, and the implantable lead further comprises a second electrical conductor extending through the second lead body lumen and mechanically and electrically coupled to the ring electrode.
In Example 12, the implantable lead of Example 11, wherein the second lead body lumen does not include a braided liner.
In Example 13, the implantable lead of any of Examples 1-12, wherein the tubular lead body comprises a polyurethane (PU) material.
In Example 14, the implantable lead of any of Examples 1-13, further comprising a proximal boot that connects the tubular lead body to the proximal connector.
In Example 15, the implantable lead of any of Examples 1-14, further comprising a distal boot that connects the tubular lead body to the distal assembly.
In Example 16, an implantable lead for use with an implantable medical device (IMD), the implantable lead comprising: a tubular lead body having a proximal end, a distal end opposite the proximal end, and a first lead body lumen extending from the proximal end through the distal end, a proximal connector at the proximal end of the lead body configured for mechanically and electrically coupling the lead to the IMD, a distal region at the distal end of the lead body including a distal assembly, the distal assembly comprising, a ring electrode positioned around an exterior of a portion of the distal region, and a helical electrode extending distally from a distal tip of the distal region; a braided liner positioned in the first lead body lumen, the braided liner including a liner lumen extending from a braided liner proximal end to a braided liner distal end, and a first electrical conductor extending through the liner lumen and electrically connected to the proximal connector and to the helical electrode.
In Example 17, the implantable lead of Example 16, wherein the braided liner comprises a plurality of ribbons that are braided together to form a tube.
In Example 18, the implantable lead of Example 17, wherein the braided liner is composed of between eight and sixty-four ribbons.
In Example 19, the implantable lead of Example 17, wherein the braided liner is composed of eight ribbons woven in a one-over-one-under pattern.
In Example 20, the implantable lead of Example 17, wherein the braided liner is woven in a one-over-one-under pattern.
In Example 21, the implantable lead of Example 17, wherein the braided liner is woven at forty-six to fifty-two pics per inch.
In Example 22, the implantable lead of Example 21, wherein each of the plurality of ribbons is composed primarily of a polyimide material or a polyethylene terephthalate (PET) material.
In Example 23, the implantable lead of Example 16, wherein the braided liner completely covers a portion of an exterior of the first electrical conductor.
In Example 24, the implantable lead of Example 16, wherein: the tubular lead body further comprises a second lead body lumen extending from the proximal end through the distal end, and the implantable lead further comprises a second electrical conductor extending through the second lead body lumen and mechanically and electrically coupled to the ring electrode.
In Example 25, the implantable lead of Example 16, wherein the tubular lead body comprises a polyurethane (PU) material.
In Example 26, the implantable lead of Example 25, wherein each of the plurality of ribbons does not contain fluoropolymer materials.
In Example 27, an implantable lead for use with an implantable medical device (IMD), the implantable lead comprising: a tubular lead body having proximal end, a distal end opposite the proximal end, and a first lead body lumen extending from the proximal end through the distal end, wherein the tubular lead body comprises a first polymer material, a proximal connector at the proximal end of the lead body configured for mechanically and electrically coupling the lead to the IMD, a distal region at the distal end of the lead body including a distal assembly, the distal assembly comprising a ring electrode positioned around an exterior of a portion of the distal region and a helical electrode extending distally from a distal tip of the distal region; a liner positioned in the first lead body lumen, the liner including a liner lumen extending from a liner proximal end to a liner distal end, wherein the liner is comprised of a second polymer material that is different from the first polymer material and a first electrical conductor extending through the liner lumen and electrically connected to the proximal connector and to the helical electrode.
In Example 28, the implantable lead of Example 27, wherein the liner comprises a plurality of ribbons that are braided together to form a tube.
In Example 29, the implantable lead of Example 27, wherein the tubular lead body comprises a polyurethane (PU) material.
In Example 30, the implantable lead of Example 29, wherein each of the plurality of ribbons does not contain fluoropolymer materials.
In Example 31, the implantable lead of Example 27, wherein the liner completely covers a portion of an exterior of the first electrical conductor.
In Example 32, the implantable lead of Example 27, wherein: the tubular lead body further comprises a second lead body lumen extending from the proximal end through the distal end and the implantable lead further comprises a second electrical conductor extending through the second lead body lumen and mechanically and electrically coupled to the ring electrode.
In Example 33, a method of manufacturing an implantable lead for use with an implantable medical device (IMD), the method comprising: braiding a plurality of ribbons to form a braided liner having a tubular shape defining a liner lumen, compressing, axially, the braided liner to increase a diameter of the liner lumen, stringing a coil conductor into the braided liner to form a conductor assembly, tensioning, axially, the conductor assembly to decrease an outer diameter of the conductor assembly, stringing the conductor assembly into a tubular lead body of the implantable lead, and releasing tension on the conductor assembly.
In Example 34, the method of Example 33, further comprising overmolding a distal boot onto a distal end of the implantable lead.
In Example 35, the method of Example 33, further comprising overmolding a proximal boot onto a distal end of the implantable lead.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram of a conduction system pacing (CSP) system, consistent with various aspects of the present disclosure.

FIG. 2 is a schematic partial cutaway side view of a lead of the CSP system of FIG. 1, consistent with various aspects of the present disclosure.

FIG. 3 is a cross-sectional view of the lead as indicated by line 3-3 in FIG. 2, consistent with various aspects of the present disclosure.

FIG. 4 is a semi-exploded view of a coil conductor with a braided liner, consistent with various aspects of the present disclosure.

FIG. 5 is a cross-sectional view of a proximal end of the lead as indicated by line 5-5 in FIG. 3, consistent with various aspects of the present disclosure.

FIG. 6 is a cross-sectional view of a distal end of the lead as indicated by line 5-5 in FIG. 3, consistent with various aspects of the present disclosure.

FIG. 7 is a flowchart of a method of manufacturing the lead, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

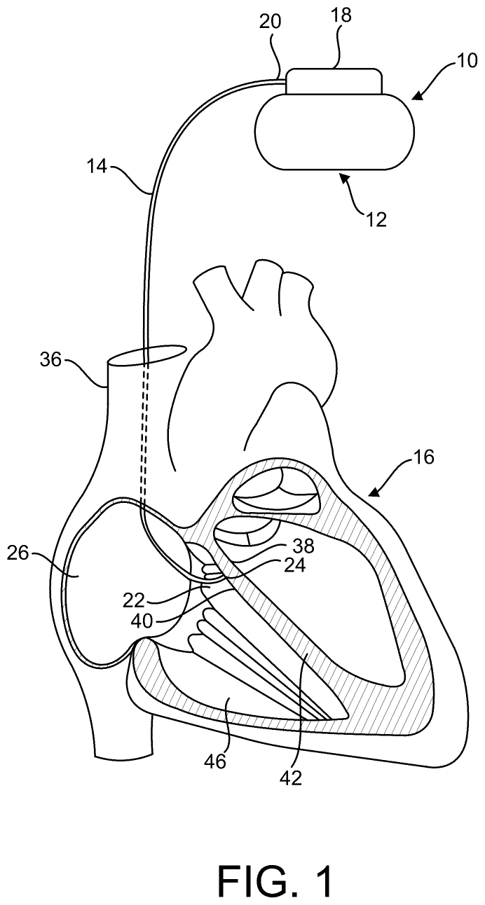

FIG. 1 is a schematic diagram of a conduction system pacing (CSP) system, according to some embodiments of this disclosure. FIG. 1 illustrates a CSP system 10 including an implantable pulse generator 12 and a lead 14. The lead 14 is implanted in a heart 16. The implantable pulse generator 12 can include circuitry for sensing bioelectrical signals and/or delivering electrical stimulation via the lead 14. The implantable pulse generator 12 can include a lead interface 18 (e.g., a header). The lead 14 can include a proximal end 20, a distal end 22, and a fixation element 24 disposed at the distal end 22.

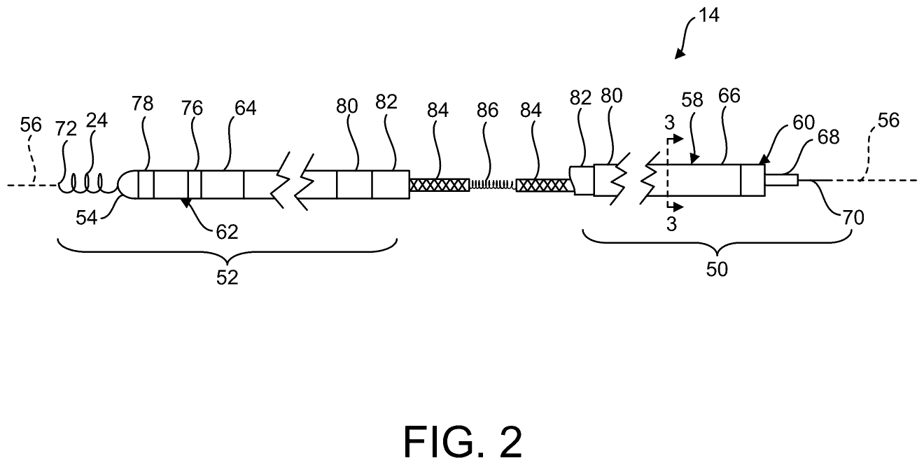

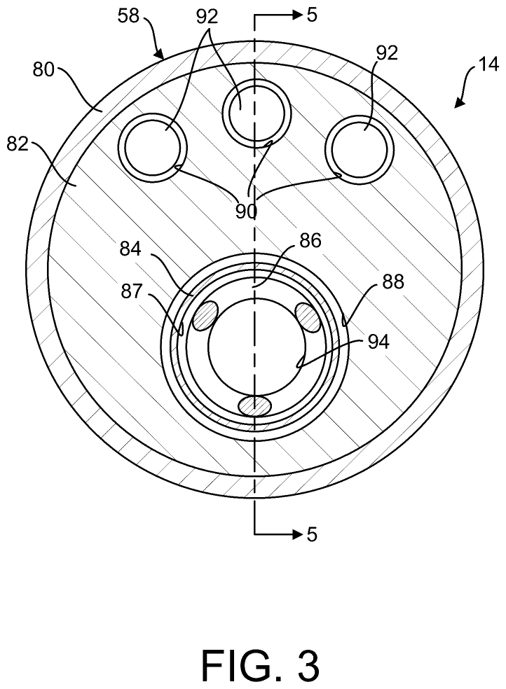

The lead 14 can further include a proximal connector having one or more electrical contacts (shown in FIG. 2) at the proximal end 20, one or more electrical elements (e.g., ring electrodes) at the distal end 22 (shown in FIG. 2), and one or more electrical conductors (e.g., one or more coils or one or more cable conductors) (shown in FIG. 2) extending within one or more lumens (shown in FIG. 3) extending within the lead 14 from the electrical contacts to the electrical elements. The lead interface 18 can connect the pulse generator 12 to the electrical contacts at the proximal end 20 of the lead 14 to electrically connect the pulse generator 12 to the electrical elements.

As shown in FIG. 1, the lead 14 is implanted in a right ventricle 46, at a ventricular septum 42, proximate a left bundle branch 38 and/or right bundle branch 40 of the specialized conduction system. The lead 14 operates to convey electrical signals between the target nerve(s), e.g., the left bundle branch 38 and/or the right bundle branch 40 and the implantable pulse generator 12. In some embodiments, the lead 14 can enter the vascular system through a vascular entry site (not shown) formed in a wall of the left subclavian vein (not shown); extend through the left brachiocephalic vein (not shown), the superior vena cava 36, and the right atrium 26; and extend to the right ventricle 46. Other suitable vascular access sites may be used in various other embodiments.

The fixation element 24 can fix the lead 14 to cardiac tissue, such as the area of tissue by which the left bundle branch 38 and/or the right bundle branch 40 can be directly stimulated. In some embodiments, the fixation element 24 can be electrically coupled to the implantable pulse generator 12 by, for example, one of the electrical conductors, such as a coil (shown in FIG. 2), extending to the proximal end 20 of the lead 14 for interfacing with the lead interface 18. As such, the fixation element 24 can mechanically and electrically couple the lead 14 to the tissue and facilitate the transmission of electrical energy from the conduction system in a sensing mode and to conduction system in a stimulation mode. In some embodiments, the fixation element 24 is a fixed fixation element, such as helix fixed to the lead 14. Such a fixation element 24 can be deployed by rotating the lead 14 itself to implant the fixation element 24 into the tissue. The use of the active fixation element for the fixation element 24 may allow for precise placement of the lead 14. The use of the active fixation element for the fixation element 24 may also provide for mapping capability because the user need not be concerned with accidental entanglement of the helix in the tissue.

While FIG. 1 only shows a single lead connected to the implantable pulse generator 12 and implanted for cardiac stimulation, various other embodiments can have an alternative lead and/or one or more additional leads for sensing bioelectrical activity and/or stimulating other areas of the heart 16.

In some embodiments, the CSP system 10 can be capable of both pacing and defibrillation therapies. In such embodiments, the lead 14 can also include one or more high voltage defibrillation electrodes (shown in FIG. 2) for delivering defibrillation shocks capable of terminating ventricular fibrillation.

FIG. 2 is a schematic partial cutaway side view of the lead 14. The lead 14 has a proximal region 50, a distal region 52 terminating at the fixation element 24 that extends from the distal tip 54, and a longitudinal axis 56. In general, the proximal region 50 is dimensioned so as to make up the portion of the lead 14 extending from the pulse generator 12 to the location at which the lead 14 enters the right atrium 26 via the superior vena cava 36, whereas the distal region 52 is dimensioned to extend within the heart 16 to the location at which the lead 14 is attached to the interior of the heart 16 (shown in FIG. 1).

As shown, the lead 14 is composed of a flexible, elongate lead body 58, a proximal connector 60, and a distal assembly 62. The flexible elongate lead body 58 generally defines the longitudinal axis 56 of the lead 14. The lead body 58 has been partially dissected in FIG. 2 to show some of the internal components thereof. For example, the outer layer 80 forms the exterior of the lead body 58, and a core 82 is positioned just inside of the outer layer 80. The core 82 includes one or more lumens (shown in FIG. 3) and a braided liner 84 is positioned inside one of the lumens. Furthermore, a coil conductor 86 is positioned inside of a liner lumen 87 of the braided liner 84, and in some embodiments, the coil conductor 86 is electrically connected to the conductor 70 and to the helical electrode 72 for transmitting electrical energy therebetween.

As further shown, the lead body 58 has a proximal end 64 and a distal end 66 opposite the proximal end 20. Additionally, the connector 60 is located at the proximal end 20 of the lead body 58, and the distal assembly 62 is located at and extends from the distal end 66 of the lead body 58. In some embodiments, the connector 60 may include a terminal pin 68 and one or more conductors 70 to electrically connect one or more active electrodes (e.g., helical electrode 72, in some embodiments) to the implantable pulse generator 12. In some embodiments, the connector 60 is a conventional bi-polar connector. In other embodiments, e.g., a quadripolar lead, the connector 60 will be configured accordingly.

In the illustrated embodiment, the lead 14 includes a shocking coil 74 and a ring electrode 76 positioned around the exterior of a portion of the distal region 52. In some embodiments, the ring electrode 76 may be entirely surrounded by insulation rendering the ring electrode 76 inactive. The ring electrode 76 is mechanically and electrically connected to the implantable pulse generator 12 by an electrical conductor (à la conductor 70) that is joined to the ring electrode 76.

The distal region 52 also includes a helical electrode 72 that extends distally from the distal tip 54 of the lead 14. In embodiments, the helical electrode 72 is configured to operate as the fixation element 24 that can be rotated into tissue in order to fix the lead 14 to a desired portion of the interior of the heart 16. Additionally, in embodiments, the helical electrode 72 is configured to be used to sense the electrical activity of the heart 16 or to apply a stimulating pulse to the cardiac tissue. This would enable a physician to use the helical electrode 72 to map cardiac tissue and thereby identify an optimal attachment site. In other embodiments, the fixation element 24 is not electrically active and merely operates as a fixation means.

The distal region 52 also includes a drug collar 78 located on the distal assembly 62. The drug collar 78 includes an exposed surface and is impregnated with a drug or therapeutic. The drug collar 78 is configured to deliver a drug or therapeutic to a desired tissue within the heart 16. In some embodiments, the drug collar 78 is an overmolded collar, and in other embodiments, the drug collar 78 is a pre-formed collar.

FIG. 3 is a cross-sectional view of the lead 14 as indicated by line 3-3 in FIG. 2. As stated previously, the core 82 includes a coil lumen 88 in which the braided liner 84 and the coil conductor 86 are positioned. The core 82 also includes other lumens 90 which can surround other elongate components 92 (e.g., stranded cable conductors for the shocking coil 74 and/or the ring electrode 76), although these elongate components 92 were omitted from FIG. 2 for the sake of clarity. A person having ordinary skill in the art would appreciate that the gaps present between the various features in FIG. 3 have been exaggerated. In some embodiments, some or all of these gaps are not present, meaning that the corresponding features are in direct contact with one another. Furthermore, while there are three lumens 90 and three elongate components 92, different numbers of lumens and components are possible.

In the illustrated embodiment, the coil conductor 86 is a trifilar coil with a central lumen 94, although in other embodiments, the coil conductor 86 has a different number of filaments (e.g., unifilar or bifilar). The central lumen 94 can accommodate other components, such as a stylet (not shown). The coil conductor 86 comprises an electrically conductive metallic material, and the coil conductor 86 is configured to conduct electrical signals, transmit torsional force (e.g., for rotating the lead 14 to implant the fixation element 24 into heart tissue), and transmit axial force (e.g., for repositioning or removal of the lead 14 from the patient). In contrast, the core 82 comprises a polyurethane (PU) material. Over time, direct contact between PU and metal can significantly weaken or degrade the PU through a mechanism known as metal ion oxidation (MIO). Thus, the lead 14 includes the braided liner 84 between the coil conductor 86 and the core 82. The braided liner 84 is configured to prevent the MIO process from occurring over time.

In some embodiments where the coil conductor 86 is not needed to conduct electricity, the core conductor 86 can be eliminated. In such embodiments, the braided liner 84 can fulfil the roles of transmitting torsional and/or axial forces through the lead body 58. In such embodiments, the size (and therefore, strength) of the braided liner 84 can be adjusted accordingly. In addition, in some embodiments, some or all of the elongate components 92 have a coiled configuration. In such embodiments, a braided liner similar to but smaller than the braided liner 84 can be positioned in the corresponding some or all of the lumens 90.

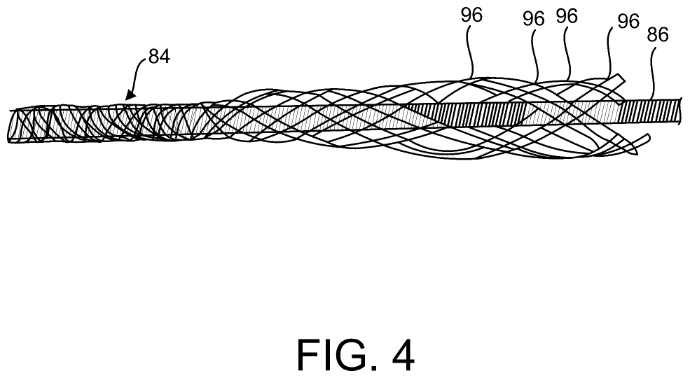

FIG. 4 is a semi-exploded view of the coil conductor 86 with the braided liner 84 surrounding the coil conductor 86. In the illustrated embodiment, the braided liner 84 has been partially unbraided and separated from the coil conductor 86 for illustrative purposes.

In the illustrated embodiment, the braided liner 84 is comprised of a plurality of ribbons 96 (although only some are labeled in FIG. 4) wound in a weave pattern to form a tube. Each ribbon 96 has the same dimensions, for example, approximately 0.0254 mm by 0.368 mm (0.001 in. by 0.0145 in.). The weave pattern can vary depending, for example, on the diameter of the coil conductor 86, which can be between approximately 0.381 mm (0.015 in.) to about 3.81 mm (0.15 in.). For some examples, if the conductor diameter is 0.762 mm (0.030 in.), then the number of ribbons 96 used will be eight weaved one-over-one-under at forty-six picks-per-inch, and if the conductor diameter is 3.05 mm (0.120 in.), then the number of ribbons 96 used will be thirty-two weaved one-over-one-under at fifty-two picks-per-inch. In such configurations, the braided liner 84 completely covers the corresponding portion of the outer surface of the coil conductor 86 that the braided liner 84 laps, which physically and chemically separates the coil conductor 86 from the core 82 (shown in FIG. 3). In other embodiments, however, the configuration of the braided liner 84 is less dense so that the braided liner 84 covers the majority (but not all) of the outer surface of the coiled conductor 86 that the braided liner 84 laps. In addition, other embodiments of the braided liner 84 with other numbers of ribbons 96 (e.g., between eight and sixty-four) and/or with differently sized ribbons 96 (or a plurality of sizes of ribbons) can be used.

In the illustrated embodiment, the braided liner 84 is able to transmit at substantial amount of axial tensile force, and the configuration and/or dimensions of the ribbons 96 can be altered to provide sufficient strength for alternative embodiments where different diameters of the coil conductor 86 are utilized. In general, altering the dimensions of the ribbons, the braid pattern, the number of ribbons in the braid, and the picks per inch (i.e., the density of the crossings of the ribbons) can alter the physical properties of the braided liner 84, for example, with respect to mechanical performance, as desired. Furthermore, making the braided liner 84 from multiple ribbons 96 increases the flexibility of the braided liner 84 compared to a liner that is a one-piece sleeve or jacket, which allows the lead 14 to remain flexible.

In the illustrated embodiment, the ribbons 96 comprise a polymer material that is not susceptible to an MIO process over the course of about ten to thirty years. In some embodiments, the ribbons 96 are composed primarily of a polyimide material. In some embodiments, the ribbons 96 are composed primarily of a polyethylene terephthalate (PET) material. Thus, in some embodiments, the ribbons 96 do not contain any fluoropolymer materials (e.g., polytetrafluoroethylene (PTFE)), so neither does the braided liner 84. Since the braided liner 84 is not susceptible to an MIO process, the braided liner 84 prevents the core 82 from MIO degradation from the coil conductor 86.

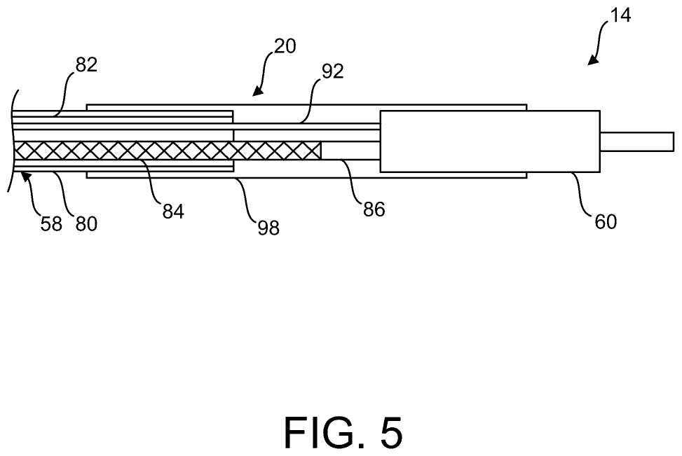

FIG. 5 is a cross-sectional view of the proximal end 20 of the lead 14 as indicated by line 5-5 in FIG. 3. In the illustrated embodiment, the outer layer 80 and the core 82 of the lead body 58 terminate distal from the connector 60, however, the coil conductor 86 and the elongate components 92 extend all the way to the connector 60. In some embodiments, the braided liner 84 extends beyond the proximal end of the core 82 but distal to the connector 60. In such embodiments, the proximal end of the braided liner 84 is captured by an overmolded boot 98, which is comprised of, for example, a high durometer polyurethane (e.g., Tecothane™) or silicone material. Such a design of the braided liner 84 can transfer an axial tension force from the terminal pin 68 to the distal end 22 (shown in FIG. 2) more directly and with reduced strain (e.g., stretching) of the lead 14. Thus, the extraction strength of the lead 14 is increased.

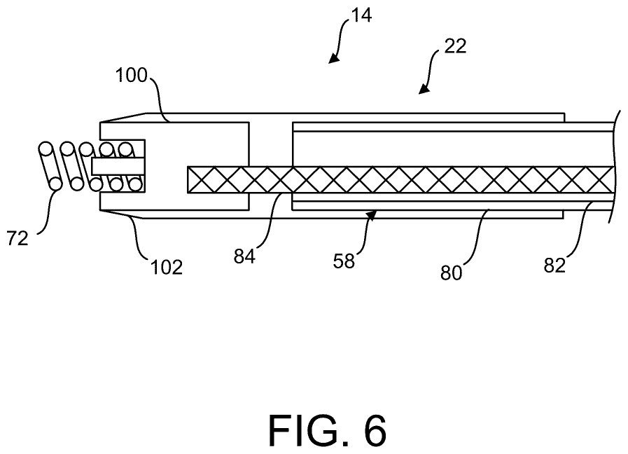

FIG. 6 is a cross-sectional view of a distal end 22 of the lead 14 as indicated by line 5-5 in FIG. 3. In the illustrated embodiment, the outer layer 80 and the core 82 of the lead body 58 terminate proximal from a coil coupler 100, However, the braided liner 84 and the coil conductor 86 (shown in FIG. 5) extend all the way into the coil coupler 100, and the coil conductor 86 is welded to the coil coupler 100 to electrically connect the coil conductor with the helical electrode 72. In some embodiments, a portion of the braided liner 84 is captured by an overmolded boot 102, which is comprised of, for example, a high durometer polyurethane (e.g., Tecothane™) or silicone material. Such a design of the braided liner 84 can transfer an axial tension force from the terminal pin 68 (shown in FIG. 5) to the distal end 22 more directly and with reduced strain (e.g., stretching) of the lead 14. Thus, the extraction strength of the lead 14 is increased.

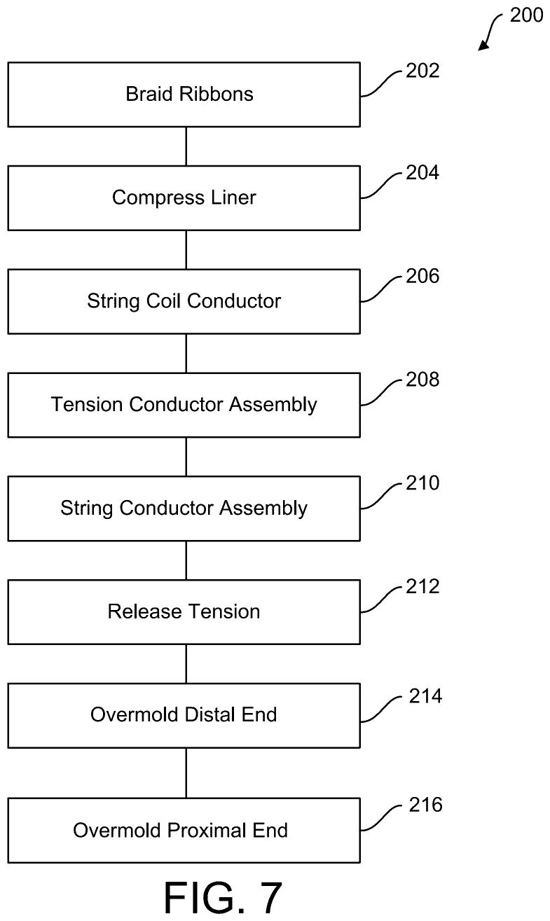

FIG. 7 is a flowchart of a method 200 of manufacturing the lead 14. The method 200 begins at operation 202, wherein the ribbons 96 are braided together to form the braided liner 84. At operation 204, the braided liner 84 is axially compressed to increase the diameter of the liner lumen 87. At operation 206, the coil conductor 86 is strung through the braided liner 84 to form a conductor assembly. At operation 208, the outer diameter of the conductor assembly is axially tensioned, for example, by pulling on the ribbons 96 and/or the coil conductor 86, to keep the conductor assembly substantially straight and to decrease the outer diameter of the conductor assembly. At operation 210, the conductor assembly is strung through the core 82.

At operation 212, the axial tension on the conductor assembly is released. At operation 214, the boot 102 is overmolded on to the lead body 58, the braided liner 84, and the coil coupler 100 to complete the distal end 22. At operation 216, the boot 98 is overmolded on to the lead body 58, the connector 60, the braided liner 84, the coil conductor 86, and the elongate components 92 to complete the proximal end 20 and, thus, the lead 14.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An implantable lead for use with an implantable medical device (IMD), the implantable lead comprising:
a tubular lead body having a proximal end, a distal end opposite the proximal end, and a first lead body lumen extending from the proximal end through the distal end;
a proximal connector at the proximal end of the lead body configured for mechanically and electrically coupling the lead to the IMD;
a distal region at the distal end of the lead body including a distal assembly, the distal assembly comprising:
a ring electrode positioned around an exterior of a portion of the distal region; and
a helical electrode extending distally from a distal tip of the distal region;
a braided liner positioned in the first lead body lumen, the braided liner including a liner lumen extending from a braided liner proximal end to a braided liner distal end; and
a first electrical conductor extending through the liner lumen and electrically connected to the proximal connector and to the helical electrode.

2. The implantable lead of claim 1, wherein the braided liner comprises a plurality of ribbons that are braided together to form a tube.

3. The implantable lead of claim 2, wherein the braided liner is composed of between eight and sixty-four ribbons.

4. The implantable lead of claim 2, wherein the braided liner is composed of eight ribbons woven in a one-over-one-under pattern.

5. The implantable lead of claim 2, wherein the braided liner is woven in a one-over-one-under pattern.

6. The implantable lead of claim 2, wherein the braided liner is woven at forty-six to fifty-two pics per inch.

7. The implantable lead of claim 6, wherein each of the plurality of ribbons is composed primarily of a polyimide material or a polyethylene terephthalate (PET) material.

8. The implantable lead of claim 1, wherein the braided liner completely covers a portion of an exterior of the first electrical conductor.

9. The implantable lead of claim 1, wherein:
the tubular lead body further comprises a second lead body lumen extending from the proximal end through the distal end; and
the implantable lead further comprises a second electrical conductor extending through the second lead body lumen and mechanically and electrically coupled to the ring electrode.

10. The implantable lead of claim 1, wherein the tubular lead body comprises a polyurethane (PU) material.

11. The implantable lead of claim 10, wherein each of the plurality of ribbons does not contain fluoropolymer materials.

12. An implantable lead for use with an implantable medical device (IMD), the implantable lead comprising:
a tubular lead body having proximal end, a distal end opposite the proximal end, and a first lead body lumen extending from the proximal end through the distal end, wherein the tubular lead body comprises a first polymer material;
a proximal connector at the proximal end of the lead body configured for mechanically and electrically coupling the lead to the IMD;
a distal region at the distal end of the lead body including a distal assembly, the distal assembly comprising:
a ring electrode positioned around an exterior of a portion of the distal region; and
a helical electrode extending distally from a distal tip of the distal region;
a liner positioned in the first lead body lumen, the liner including a liner lumen extending from a liner proximal end to a liner distal end, wherein the liner is comprised of a second polymer material that is different from the first polymer material; and
a first electrical conductor extending through the liner lumen and electrically connected to the proximal connector and to the helical electrode.

13. The implantable lead of claim 12, wherein the liner comprises a plurality of ribbons that are braided together to form a tube.

14. The implantable lead of claim 12, wherein the tubular lead body comprises a polyurethane (PU) material.

15. The implantable lead of claim 14, wherein each of the plurality of ribbons does not contain fluoropolymer materials.

16. The implantable lead of claim 12, wherein the liner completely covers a portion of an exterior of the first electrical conductor.

17. The implantable lead of claim 12, wherein:
the tubular lead body further comprises a second lead body lumen extending from the proximal end through the distal end; and
the implantable lead further comprises a second electrical conductor extending through the second lead body lumen and mechanically and electrically coupled to the ring electrode.

18. A method of manufacturing an implantable lead for use with an implantable medical device (IMD), the method comprising:
braiding a plurality of ribbons to form a braided liner having a tubular shape defining a liner lumen;
compressing, axially, the braided liner to increase a diameter of the liner lumen;
stringing a coil conductor into the braided liner to form a conductor assembly;
tensioning, axially, the conductor assembly to decrease an outer diameter of the conductor assembly;
stringing the conductor assembly into a tubular lead body of the implantable lead; and
releasing tension on the conductor assembly.

19. The method of claim 18, further comprising overmolding a distal boot onto a distal end of the implantable lead.

20. The method of claim 18, further comprising overmolding a proximal boot onto a distal end of the implantable lead.
